(19) United States
(12) Patent Application Publication
(10) Pub. No.: US 2022/0401511 A1
(43) Pub. Date: Dec. 22, 2022

(54) ANTI-AGING DIETARY SUPPLEMENT

(71) Applicants: Brent D. Willis, American Fork, UT (US); Fernando Swartz, American For, UT (US); Afa K. Palu, American Fork, UT (US); Brett J. West, American Fork, UT (US)

(72) Inventors: Brent D. Willis, American Fork, UT (US); Fernando Swartz, American For, UT (US); Afa K. Palu, American Fork, UT (US); Brett J. West, American Fork, UT (US)

(21) Appl. No.: 17/349,051

(22) Filed: Jun. 16, 2021

(51) Int. Cl. A61K 36/9066; A61K 36/82; A61K 36/45; A61K 36/746; A61K 31/198; A61K 31/4045; A61K 31/455; A61K 31/4415; A61K 31/714; A61K 36/55; A61K 36/87; A61K 33/30; A61K 33/04; A61K 33/24; A61K 33/06; A61K 31/197; A61K 9/00

(52) U.S. Cl. CPC A61K 36/9066; A61K 36/82; A61K 36/45; A61K 36/746; A61K 31/198; A61K 31/4045; A61K 31/455; A61K 31/4415; A61K 31/714; A61K 36/55; A61K 36/87; A61K 33/30; A61K 33/04; A61K 33/24; A61K 33/06; A61K 31/197; A61K 9/0053

(57) ABSTRACT

The present invention relates to a formulation providing anti-aging benefits that may include ingredients from various ingredient groups such as amino acids, vitamins or mineral co-factors, antioxidants or carotenoids, an herbal, plant derivative, or phytonutrients, and other nutrients. When combined, these ingredients and, more specifically, the herbal, plant derivative, or phytonutrients blend provide increased and unexpected synergistic effects that inhibit DPP4 activity and provide anti-aging benefits.

### BACKGROUND

### 1. Field of Invention

The present invention relates to an anti-aging dietary supplement. More specifically, the present invention relates to methods and compositions that provide improved anti-aging effects using a combination of ingredient groups.

### 2. Background

The aging process is a relentless fact of human life and health. Human aging can be caused by multiple factors, including chronic disease occurrence, cellular senescence, diminished homeostatic control, disruption in DNA repair, reduction in growth hormone levels, inefficient energy metabolism, mitochondrial dysfunction, and oxidative damage affecting structures from molecules to entire organ systems. Sometimes these factors are due to genetics, body physiology, biochemistry, lifestyle behaviors, poor sleep, and even external factors such as air pollution, occupational exposures, harsh environments, smoking, and hazardous events.

Inevitably, aging can adversely affect cognition, sensory function, cardiovascular health, kidney and lung function, immune system competence, and bone and muscle integrity. Current anti-aging treatments include molecular repair, increasing telomerase activity, cellular rejuvenation, cloning, replacement of damaged stem cells, tissues, and organs. However, these therapeutic treatments can often require surgery, parenteral injections, cosmetics, and stem cells. This is undesirable because these treatments are relatively invasive and may have significant side effects.

### SUMMARY OF THE INVENTION

Embodiments of the present invention relate to an anti-aging dietary supplement. More specifically, embodiments of the present invention relate to methods and compositions that provide improved anti-aging effects by using a combination of ingredient groups.

The production and release of growth hormone (“GH”) in the bloodstream can be beneficial in delaying the effects of aging. An enzyme, dipeptidyl peptidase-4 (“DPP4”), can reduce GH release and, in doing so, encourage the aging process. Therefore, DPP4 inhibitors can delay the aging process by increasing the production and release of GH into the blood.

In some embodiments, the anti-aging supplement includes ingredients from various ingredient groups such as amino acids, vitamins or mineral co-factors, antioxidants or carotenoids, an herbal, plant derivative, or phytonutrients, and other nutrients. In some embodiments, the herbals, plant derivatives, and phytonutrient ingredient group (also referred to as an “herbal blend”) may include noni fruit juice, green tea extract, grape seed extract, curcumin, blueberry fruit extract, or any combination thereof. Each phytonutrient, or phytochemical, is, individually, a DPP4 inhibitor. But, as discovered through experimentation, when some of these ingredients are combined, the collective—or synergistic—effects yield surprising results and unexpected advantages.

In one embodiment, the herbal blend, including curcumin, grape seed extract, green tea, and noni, can provide an actual DPP4 inhibition of 96%. This is 31% above expectations based on individual ingredient test results. In another embodiment, the herbal blend including curcumin, blueberry, green tea, and noni can provide an actual DPP4 inhibition of 99.9%. This is 49% above expectations based on individual ingredient test results. This shows an unexpected, synergistic effect when specific ingredients, or ingredient groups, are combined that yield improved DPP4 inhibition percentages well beyond expectations. Additionally, these specific herbal blends yield more powerful effects related to GH releasing potential while requiring considerably lower concentrations of each ingredient.

### BRIEF DESCRIPTION OF THE DRAWINGS

In order that the matter in which the above-recited and other advantages of the invention are obtained, a more particular description of the invention briefly described above will be rendered by reference to specific embodiments thereof which are illustrated in the appended drawings. Understanding that these drawings depict only typical embodiments of the invention and are not, therefore, to be considered to be limiting of its scope, the invention will be described and explained with additional specificity and detail through the use of the accompanying drawings in which:

FIG. 1 depicts Table 1, showing Example Formulation Ranges;

FIG. 2 depicts Table 2, showing an Example Formulation;

FIG. 3 depicts Table 3; and

FIG. 4 depicts Table 4, showing Example One Test Results.

### DETAILED DESCRIPTION OF THE INVENTION

It will be readily understood that the components of the present invention, as generally described herein, could be arranged and designed in a wide variety of different configurations. Thus, the following more detailed description of embodiments of the compositions and methods of the present invention is not intended to limit the scope of the invention, as claimed, but is merely representative of the presently preferred embodiments of the invention. Therefore, the scope of the invention is indicated by the appended claims rather than by the foregoing description. All changes that come within the meaning and range of equivalency of the claims are to be embraced within their scope.

Embodiments of the present invention relate to an anti-aging dietary supplement that derives its benefits from multiple mechanisms of action. In particular, one featured mechanism of action is the promotion of growth hormone (“GH”) in the bloodstream. The production and release of GH in the bloodstream can be beneficial in delaying the effects of aging. However, an enzyme, dipeptidyl peptidase-4 (“DPP4”), can reduce GH release and, in doing so, encourage the aging process. Therefore, DPP4 inhibitors can promote anti-aging by increasing the production and release of GH into the blood. In addition to delaying the aging process, DPP4 inhibitors can also treat type-2 diabetes and control blood glucose levels. DPP4 degrades incretions, a group of hormones that stimulate insulin release from pancreatic beta cells and inhibit release from pancreatic beta cells. As such, DPP4 inhibitors are capable of lowering blood glucose levels. Lower blood glucose concentrations stimulate GH release from the pituitary. Thus, inhibiting DPP4 is capable of promoting GH release by at least two mechanisms of action.

In some embodiments, the present invention relates to methods and compositions that inhibit DPP4 activity and promote anti-aging benefits. In some embodiments, the formulations may include ingredients from the following ingredient groups: amino acids, vitamins, mineral co-factors, antioxidants, carotenoids, phytonutrients from herbal sources, plant products, or their derivatives, and other nutrients adapted for human consumption to optimize protective effects against the aging process. In some embodiments, the formulation may include ingredients from only a single category, ingredients from multiple categories, may not include any ingredients from any particular category, or any combination thereof.

In some embodiments, the herbals, plant derivatives, and phytonutrients category (also referred to as an “herbal blend”) may include Vaccinium corymbosum (“blueberry”), Curcuma longa (“turmeric”) root extract (comprising curcumin), Vitis vinifera (“grape”) seed extract, Camellia sinensis leaf (“green tea”) extract, and Morinda citrifolia (“noni”) fruit juice, or any combination thereof. For example, in some embodiments, the herbal blend may include curcumin, grape seed extract, green tea, and noni. In other embodiments, the herbal blend may include curcumin, blueberry, green tea, and noni.

Each phytonutrient, or phytochemical, is a DPP4 inhibitor and can increase GH in the bloodstream. This increase of GH in the bloodstream can contribute to anti-aging benefits. But, when specific ingredients are combined, the collective—or synergistic—effects yield unexpected and improved benefits. For example, the herbal blend including curcumin, grape seed extract, green tea, and noni dramatically and unexpectedly improves DPP4 inhibition. Based on individual ingredient test results, the expected percent DPP4 inhibition of this herbal blend was only 65%. But, based on experimental results presented below, the actual percent DPP4 inhibition of this herbal blend was 96%. This shows a synergistic effect when specific ingredients, or ingredient groups, are combined that yield DPP4 inhibition percentages well beyond expectations—specifically, 31% above what is expected.

In another example, the herbal blend including curcumin, blueberry, green tea, and noni also dramatically and unexpectedly improved DPP4 inhibition. Based on individual ingredient test results, the expected percent DPP4 inhibition of this herbal blend was only 51%. But, based on experimental results presented below, the actual percent DPP4 inhibition of this herbal blend was 99.9%. This further shows an unexpected synergistic effect when specific ingredients, or ingredient groups, are combined that yield improved DPP4 inhibition percentages—specifically, 49% above what is expected. Additionally, these specific herbal blends yield significantly more powerful effects as it relates to GH releasing potential while requiring considerably lower concentrations of each ingredient.

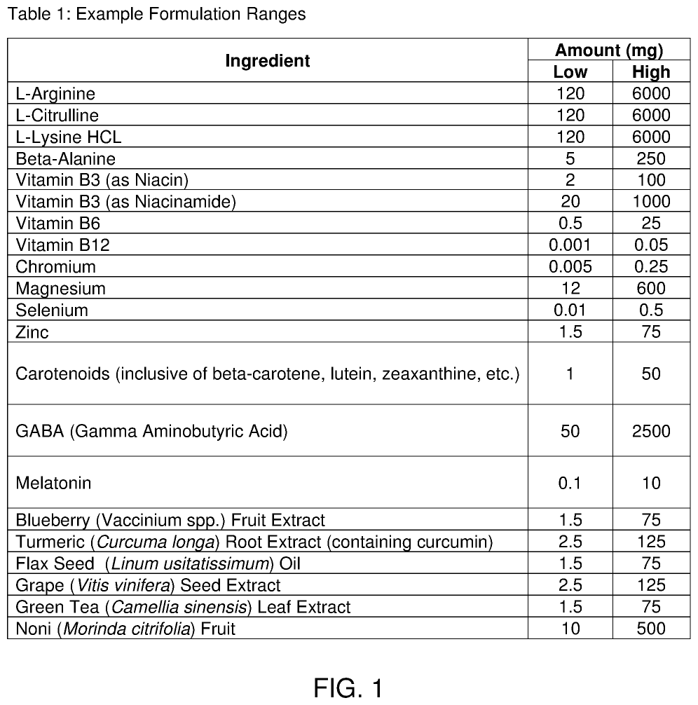

FIG. 1 depicts Table 1, showing, in one embodiment, composition ranges of the present invention. The composition ranges are shown in milligrams (“mg”) by weight of the formulation. For example, in some embodiments, L-Arginine, L-Citrulline, and L-Lysine HCL may each be present in an amount between about 120 and 6000 mg. In some embodiments, beta-alanine may be present in an amount between about 5 and 250 mg. In some embodiments, Vitamin B3 (as Niacin) may be present in an amount between about 2 and 100 mg. In other embodiments, Vitamin B3 (as Niacinamide) may be present in an amount between about 20 and 1000 mg. In other embodiments, Vitamin B6 may be present in an amount between about 0.5 and 25 mg. In some embodiments, Vitamin B12 may be present in an amount between about 0.001 and 0.05 mg. In some embodiments, chromium may be present in an amount between about 0.005 and 0.25 mg. In some embodiments, magnesium may be present in an amount between about 12 and 600 mg. In other embodiments, selenium may be present in an amount between about 0.01 and 0.5 mg. In yet other embodiments, zinc, blueberry (Vaccinium spp.) fruit extract, flaxseed (Linum usitatissimum) oil, and green tea (Camellia Sinensis) leaf extract may each be present in an amount between about 1.5 and 75 mg. In some embodiments, at least one carotenoid (such as beta-carotene, lutein, zeaxanthin, etc.) may be present in an amount between about 1 and 50 mg. In some embodiments, only a single carotenoid is present, while in other embodiments, more than one carotenoid may be present—creating a mixture of carotenoids. In some embodiments where a mixture of carotenoids is present, the weight of the formulation in mg represents the total weight of the carotenoid mixture. In some embodiments, turmeric (Curcuma longa) root extract (comprising curcumin), and grape (Vitis vinifera) seed extract, may each be present in an amount between about 2.5 and 125 mg. In some embodiments, GABA (gamma-aminobutyric acid) may be present in an amount between about 50 and 2500 mg. In other embodiments, melatonin may be present in an amount between about 0.1 and 10 mg. In still other embodiments, noni (Morinda citrifolia) fruit may be present in an amount between about 10 and 500 mg.

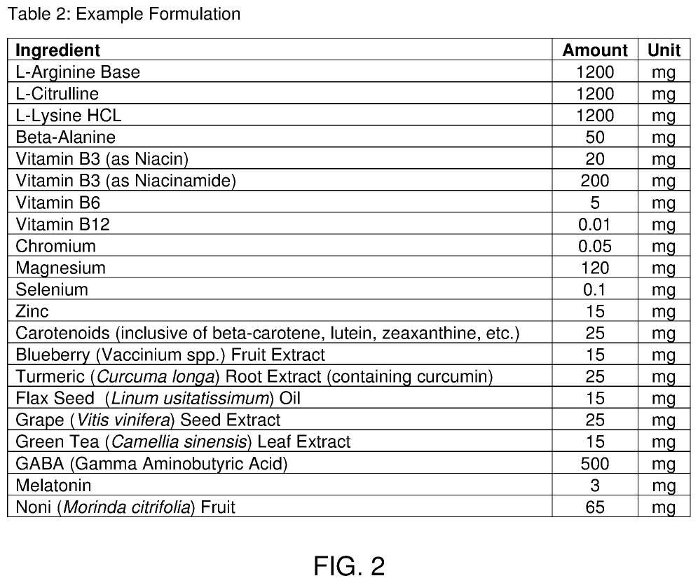

FIG. 2 depicts Table 2, showing, in one embodiment, an example formulation of the present invention. The example formulation lists the ingredients in mg by weight of the formulation. For example, in some embodiments, L-Arginine, L-Citrulline, and L-Lysine may each be present in an amount equal to 1200 mg. In some embodiments, beta-alanine may be present in an amount equal to 50 mg. In other embodiments, Vitamin B3 as Niacin may be present in an amount equal to 20 mg. In some embodiments, Vitamin B3 as Niacinamide may be present in an amount equal to 200 mg. In some embodiments, Vitamin B6 may be present in an amount equal to 5 mg. In other embodiments, Vitamin B12 may be present in an amount equal to 0.01 mg. In still other embodiments, chromium may be present in an amount equal to 0.05 mg. In some embodiments, magnesium may be present in an amount equal to 120 mg. In some embodiments, selenium may be present in an amount equal to 0.1 mg. In yet other embodiments, zinc, Vaccinium corymbosum fruit extract, Linum usitatissimum oil, and Camellia sinensis leaf extract may each be present in an amount equal to 15 mg. In some embodiments, at least one carotenoid may be present in an amount equal to 25 mg. In some embodiments, only a single carotenoid is present, while in other embodiments, more than one carotenoid may be present—creating a mixture of carotenoids. In some embodiments where a mixture of carotenoids is present, the weight of the formulation in mg represents the total weight of the carotenoid mixture. In some embodiments, Curcuma longa root extract comprising curcumin, and Vitis vinifera seed extract, may be present in an amount equal to 25 mg. In some embodiments, GABA may be present in an amount equal to 500 mg. In other embodiments, melatonin may be present in an amount equal to 3 mg. And still, in other embodiments, Morinda citrifolia fruit may be present in an amount equal to 65 mg.

In one embodiment, the formulation may include four or more ingredients in the amino acids category, one or more of the ingredients in the antioxidants and carotenoids category, three or more ingredients in the herbals, plant derivatives, and phytonutrients category, and optionally, one of the ingredients in the other nutrient category.

In one embodiment, the primary dosing and ranges of the dietary supplement are designed to be at broad and at sufficient levels to have the desired beneficial anti-aging effects in humans. The primary classes of micro/phytonutrients to which the components of the present formulation belong are known to enhance growth hormone levels, reduce oxidative damage and inflammation, support immune system function and neuroprotection and enhance optimal health during aging.

Embodiments of this invention can relate to any form of administration of these formulations in humans, including tablets, bars, liquids, powders, gummies, lozenges/troches, dissolvable disks, chewable, inhalational forms, injectables, patches, ointments, gels, aerosols, aerosols, and sticks as well as any transcutaneous, intranasal, intra-cavitary, liposomal nanotechnology or other delivery systems.

### Category and Ingredient Rationale

This section describes the scientific rationale for using components that may be present in the current invention. The following categories are Amino Acids, Vitamins and Mineral Co-Factors, Antioxidants and Carotenoids, and Herbals, Plant Derivatives, and Phytonutrients.

1. Amino Acids

Amino acids can be beneficial in anti-aging formulations because of their biological characteristics. In particular, amino acids can benefit anti-aging formulations because they make up the largest component in body tissues, besides water. In addition, amino acids may be categorized as essential, non-essential (the body can manufacture them), or conditional (the body cannot produce them during stress or illness). In some embodiments of the present invention, all three types of amino acids may be present.

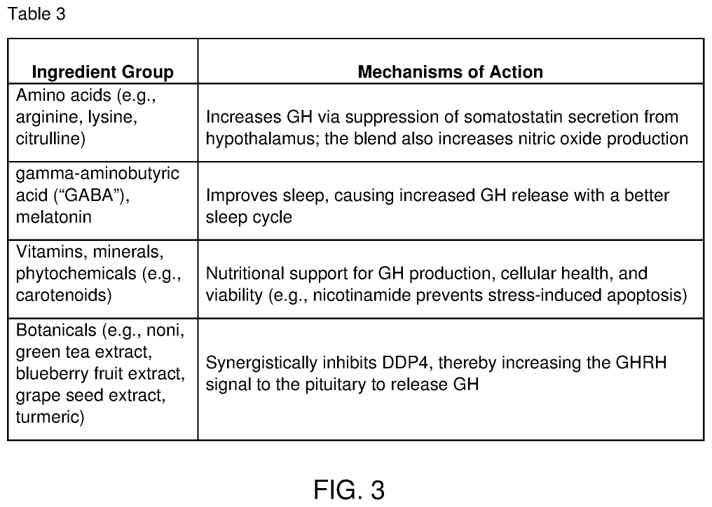

Chains of amino acids make up peptides which are bioactive compounds that have beneficial metabolic effects. As shown in FIG. 3, chains of amino acids can also boost GH levels as well as substances that maintain optimal pH balance. Unbranched chains of several peptides are known as polypeptides, and when longer than approximately 50 amino acids, they are designated as a protein. A bioactive protein is a “straight” polypeptide chain folded and optically arranged into a three-dimensional biologically functional configuration. Besides protein formation, these “building blocks” can be essential for neurotransmitter function, immune system integrity, energy production, antioxidant defense, and nitric oxide-related molecular signaling for physiological processes.

L-Arginine is the direct substrate for the family of nitric oxide synthases and is involved in metabolic processes of immune cells, including T-cell growth and lymphocyte function. This amino acid can also increase nitric oxide production with the resultant promotion of vascular dilation and reduced LDL-cholesterol levels and platelet and white blood cell adhesion. It is further anticipated that muscle strength and recovery can be improved, thereby providing ergogenic benefits. These results have been demonstrated in human studies where exertion-related oxygen consumption was reduced and exercise tolerance enhanced. In addition, experimental models show that oxidative damage can be decreased, immune function increased, and organ function improved. Amino acid mixtures such as the Arginine pyroglutamate form, which may be used in the present invention, can increase solubility and stability, are inherently non-toxic, and enhance biopharmaceutic effects.

L-Arginine and L-Citrulline may be synergistic in these effects, including in untrained yet healthy individuals. In some embodiments, L-Citrulline may be included in the present invention and is described in more detail below. The potential positive effects from this combination of amino acids may be beneficial for mitochondrial disease and psychological depressive states. In connection to delaying the aging process, L-Arginine and L-Lysine, alone and in combination, can have anti-inflammatory and bone health benefits.

The amino acid beta-alanine can be produced by the body (referred to as “non-essential”). Beta-alanine is not involved in protein accretion but is the only naturally occurring beta-amino acid. Beta-alanine provides its most significant benefits by enhancing the synthesis of the peptide carnosine and providing adjunctive effects to L-Arginine. Carnosine is a precursor to nitric oxide synthase generation and subsequent nitric oxide production. It may improve cognition and optimize muscular acid-base balance, thereby increasing strength, mass, and endurance while decreasing fatigue. As stated previously, Arginine is critical in nitric oxide production, energy metabolism, and immune function and reduces inflammation and harmful low-density lipoprotein profiles.

L-Citrulline is closely related to the metabolism of L-Arginine. Since L-Citrulline is not subject to hepatic degradation, it is metabolized into Arginine and can effectively enhance available levels of Arginine in the body. Additionally, L-Citrulline can play a role in protein synthesis and nitric oxide metabolism. The effects of increased nitric oxide on human performance have been extensively evaluated. In clinical trials of healthy male athletes, performance during exertion and recovery has been positively influenced by L-Citrulline. This shows that L-Citrulline may aid in the preservation of functional capacity as individuals age. In addition, the vascular effects of this amino acid can have a therapeutic impact on heart disease and cold exposure stress. Clinical and experimental situations indicate that these benefits may extend to the elderly. Finally, L-Citrulline exhibits beneficial influences in ischemic conditions and liver damage.

The amino acid L-Lysine may be used as a food additive. It has been shown to have several critical functions, including anti-viral effects, support of collagen production, reduction of anxiety, and beneficial metabolic effects. L-Lysine and its partner L-Arginine balance are also significant since the latter amino acid enhances immune function through antibody development. Two other factors related to Lysine may be significant in terms of this invention. First, L-Lysine has an adjunctive effect in the production of carnitine, that among other benefits, as noted above, lowers cholesterol levels and converts fatty acids into energy. Second, Lysine also improves the efficacy of L-Arginine in promoting human GH release, which is one benefit of the present invention.

2. Vitamins and Mineral Co-Fact

As shown in FIG. 3 depicting Table 3, Vitamins and minerals can support GH production along with cellular health and viability. Vitamin B3, or Niacin, is a water-soluble vitamin that exists in nicotinic acid and amide nicotinamide form—each with different functions. This B vitamin is a component of key coenzymes necessary for cellular metabolism and energy production and a role as an antioxidant in cell signaling and DNA synthesis and repair. The acid form of Niacin improves lipid profiles and provides anti-inflammatory effects, both of which are vascular protective. The amide form of Vitamin B3 is efficacious in photoprotection and chemoprevention from skin disease and osteoarthritis, two conditions mainly related to the aging process.

Niacinamide is another form of Niacin, consisting of a pyridine nucleoside configuration of the parent Vitamin B3. In some embodiments, it can contribute to anti-aging because it functions as a precursor to nicotinamide adenine dinucleotide (“NAD”), a coenzyme vital in energy metabolism, brain mitochondrial function, and biological processes. NAD levels drop increasingly as individuals age.

Vitamin B6 can provide benefits to the health of the nervous and immune systems. Specifically, this vitamin is involved in the biosynthesis of neurotransmitters, thereby impacting cognitive development. Deficiency is associated with several clinical conditions resulting in low plasma concentrations. While adequate levels have not definitively shown effect in cancer, there may be some benefit in cardiovascular disease and neurocognitive domains.

Vitamin B12 is a water-soluble vitamin that is necessary for neurological function, DNA synthesis, and red cell formation and acting as an important enzymatic co-factor. As with other B vitamins, deficiency syndromes can show a wide spectrum of symptoms. Older individuals are especially affected. Because of its cognitive effects, Vitamin B12 continues to be assessed in dementias.

The chemical element chromium has widespread applications, including in human health. The trivalent ionic form is thought to be a beneficial nutrient because of its impact on lipid and glucose metabolism that are known to be altered with aging. This trace element has a mechanistic role in the action of insulin, thereby affecting protein, carbohydrate, and fat metabolism. The chromium content in foods is generally low, so supplementation may be a reasonable strategy and recommended adequate intakes have been established. Qualified health claims for chromium have been allowed in the U.S. and Canada as a factor in the maintenance of good health.

Magnesium holds a prominent position among essential elements that function as required co-factors for critical enzymatic reactions, biochemical function, and metabolic pathways. Its supplementation is generally safe and is an effective therapeutic intervention in many clinical situations. It may also have a role as an electrolyte. This mineral has been shown to be beneficial in regard to decreasing inflammatory responses and reducing complications in high-risk surgery.

Selenium is a co-factor of glutathione peroxidase, and Se-glutathione peroxidase also acts as an antioxidant. This mineral also decreases the harmful effects of pro-inflammatory cytokines and is radio-protective. Recent experimental studies have shown that selenium may protect against DNA damage and inhibit tumor progression. It is also involved in the activity of superoxide dismutase, providing further cellular defense against reactive oxygen species. Therefore, selenium supplementation at adequate but safe doses is essential for promoting antioxidant enzyme systems and reducing potential oxidative stress.

As has been emphasized in this section, trace elements play an important role in human health. Zinc is an essential micronutrient in this regard and functions as a biological catalyst and regulator with critical effects in oxidative stress, immune function, cellular integrity, and the aging process. Since zinc levels decline with age, there are broad international efforts to ascertain appropriate supplementation guidelines. In this population, zinc is vital to the maintenance of DNA repair and genomic stability. Zinc enhances superoxide dismutase activity and provides a protective antioxidant function against peripheral neuropathy in diabetic animal models. Several chronic diseases related to oxidative stress and inflammation are associated with inadequate zinc levels.

3. Antioxidants and Carotenoids

Intracellular antioxidants such as glutathione are important because of their immune system defense and provide benefits to neurologic pathophysiology. Glutathione can catabolize anions and hydrogen peroxide and is a potent intracellular protective agent against excess free radical damage. However, since it is hydrolyzed in the intestine and generally ineffective when consumed orally, this invention takes advantage of alternative anti-aging strategies against oxidative damage.

Although natural-source mixed carotenoids usually contain at least four carotene configurations (alpha, beta, gamma, delta), beta-carotene is the most con on and dominant form. In the body, beta-carotene is a vitamer (similar molecular structure) and precursor of vitamin A. This carotenoid is an essential antioxidant that decreases free radical-induced oxidative stress. Vitamin A is critical for the induction of beneficial cellular differentiation. At the same time, beta-carotene increases the connexin gene expression, which encodes for a gap junction protein that is necessary for maintaining normal cellular phenotypes. It is also more effective in quenching oxygen-derived free radicals. These antioxidants protect cellular components in lipid and high oxygen environments, but only the natural form of beta-carotene can effectively decrease radiation-induced cellular abnormalities. In combination with other antioxidants, beta-carotene is an effective radioprotectant and can address ischemia and reperfusion injury, biological damages relevant to aging.

Other dietary carotenoids are equally important. Similar protection against oxidative stress and radiation is noted in vitro using lycopene, a potent antioxidant in tomato juice. This compound also reduces oxidative damage related to physical activity. In addition, relevant to the aging process, it protects against neurotoxicity and provides biochemical stabilization in brain injury models.

Lutein is another dietary carotenoid that demonstrates antioxidant properties in humans with related health benefits. It functions as a strong antioxidant in vivo and reduces oxidative stress in individuals with low antioxidant status subjected to hostile environments. This substance has a unique characteristic in that it can induce increases in physical activity in sedentary persons and is also neuroprotective against transient brain ischemia. Relative to the current invention, multiple antioxidants are virtually always more effective than the individual agents themselves, such as noted in tumor cell growth inhibition.

4. Herbals, Plant Derivatives, and Phytonutrients

Some species of blueberry fruits (genus Vaccinium) contain polyphenols and anthocyanidin molecules which are powerful antioxidant compounds. These phytochemicals—also referred to as phytonutrients—are readily soluble and bioavailable in many forms. Their bioactivity in terms of free radical scavenging and anti-inflammatory effect has been well documented. As an anti-aging strategy, the interplay between inflammation and obesity may provide another intervention target for blueberry fruits. Blueberry fruits can provide cardiometabolic-related improvement in lipid status, vascular function, and nitric oxide vasodilatory bioactivity. In addition, this phytonutrient is potentially beneficial as an anti-cancer radiosensitizer. Blueberry polyphenols and anthocyanins can function as neuroprotective agents, causing memory enhancement and neurocognitive improvement.

Curcumin is a phytonutrient abundantly present in the spice turmeric and demonstrates a broad spectrum of therapeutic properties. It is potentially effective to lung protection from hazardous exposures has been described. Immune enhancement with antibacterial activity is another potential benefit. Curcumin has an important impact on cognitive decline through its antioxidant and anti-inflammatory actions and its contribution to neural plasticity and repair, hippocampal neurogenesis, and amyloid beta-protein plaque binding. This polyphenolic agent decreases muscular pain and reduces inflammation and post-exercise muscle damage in human subjects. New highly absorbable formats may also benefit bone health. It improves in vivo neural functioning and protects against oxidative stress and brain ischemia-reperfusion injury. Curcumin has also shown benefit against oxidative damage, and in combination with resveratrol, demonstrates unique radio-protective qualities on normal tissues.

Grape seeds and extracts thereof protect against ultraviolet radiation via their phenolic compounds, the proanthocyanidins. Grape juice polyphenols and resveratrol improve antioxidant capacity and diminish oxidative stress. These compounds protect neurocognitive function in older adults and spatial memory and performance in younger adults. Resveratrol is the predominant polyphenolic compound derived from the skin of grapes and other berries and is also neuroprotective as a single agent. It can be beneficial in environmental exposure, as shown by its protective effect against the oxidative stress caused by cigarette smoke. In addition, it can provide a protective antioxidant effect against radiation damage. In an adjunctive manner, the current invention may utilize compounds chemically related to resveratrol such as pterostilbene. Pterostilbene is a naturally derived primary antioxidant component of blueberries with greater oral absorption and, through lipophilic function, greater bioavailability than resveratrol. It has demonstrated antioxidant, anti-inflammatory, biological, and anticarcinogenic properties in experimental and in vivo models. However, while resveratrol is Generally Recognized as Safe (“GRAS”) for chronic use, pterostilbene's safety and efficacy have not been clinically evaluated long enough for full assessment or GRAS status designation.

Green and black tea beverages have had strong antioxidant effects in human trials and in vitro protection against radiation exposure. It has also been suggested that green tea, in combination with other phytonutrients, may provide substantial photoprotection. Extracts of black and green tea, including the compound theanine, have neuroprotective qualities. These components prevent memory impairment and improve cognitive function in humans of varying ages, and show promising effects in psychiatric and neurodegenerative disorders.

Morinda citrifolia, commonly known as noni, possesses a wide range of biological activities that are beneficial to human health. Human intervention studies demonstrate that noni fruit juice provides protection against tobacco smoke-induced DNA damage, blood lipid, and homocysteine elevation, as well as systemic inflammation. Human intervention studies also reveal that noni juice improves joint health, increase physical endurance, increase immune activity, inhibit glycation of proteins, aid weight management, help maintain bone health in women, help maintain normal blood pressure, and improve gum health. Further, these studies reveal that noni juice exhibits significant antioxidant activity, more so than other fruit juices, which served as trial placebos. The antioxidant effect of noni juice and its interaction with the immune system and inflammation pathways account for many of the observed health benefits of noni juice. However, several other biological processes of the human body are also positively influenced by noni juice.

A unique and unexpected aspect of the present invention, as discussed above and shown in FIG. 3 depicting Table 3, is the synergistic effect of the herbal blend ingredients that inhibit DPP4 at increased percentages. The hypothalamus produces growth hormone-releasing hormone (“GHRH”). GHRH stimulates the pituitary gland to produce and release GH into the bloodstream. DPP4 is an enzyme that breaks down GHRH. Therefore, inhibitors of DPP4 can help increase GH release into the blood.

Morinda citrifolia (noni) fruit juice has been shown to inhibit DPP4 in vitro. Camellia sinensis leaf (green tea) extract, Vitis vinifera (grape) seed extract, Vaccinium corymbosum (blueberry), and Curcuma longa (turmeric) extract (comprising curcumin) also inhibit DPP4 activity in vitro. But, when these ingredients are combined and tested together, the resulting DPP4 inhibition is approximately 31% to 48.44% more than expected, as shown in the example below. This indicates a unique synergistic effect towards DPP4 inhibition by creating a more powerful impact related to GH releasing potential when DPP4 inhibiting action is increased. DPP4 can also degrade glucagon-like peptide-1 (GLP-1), which helps decrease blood glucose levels by stimulating insulin secretion from pancreatic β cells. Low glucose levels can also stimulate GH release from the pituitary. Therefore, DPP4 inhibitors can influence circulating GH levels through at least two biological pathways.

### Method of the Invention

Methods of the present invention consider the unique pharmacokinetics, pharmacodynamics, and nutritional properties of the ingredients. Their collective biological activities contribute to the production of GH and its secretion from the pituitary. In some embodiments, the dietary supplement can be administered via capsule, powder, or liquid delivery format. This type of administration may be consumed daily. The following disclosure of the present invention is grouped into subheadings. The utilization of the subheadings is for the convenience of the reader only and is not to be construed as limiting in any sense.

### Examples

The following example illustrates some of the embodiments of the present invention. These examples are not intended to be limiting in any way. They are merely illustrative of the benefits, advantages, and remedial effects of some embodiments of the compositions of the present invention.

As illustrated by the following example, embodiments of the present invention have been tested. Specifically, the example illustrates the results from ingredient testing have unexpected benefits as it relates to GH releasing potential by inhibiting DPP4 at much greater percentages than expected.

### Test One

The DPP4 inhibiting effects of individual ingredients and combinations of ingredients were evaluated in vitro and directly with the DPP4 enzyme, as described below. The percentage of concentration refers to the concentration strength of the particular concentrate tested; that is, the strength of concentration relative to the processed product from which the concentrate was obtained.

### Research Method

Noni fruit juice from puree was filtered through a 0.45 μm filter and then twice diluted to prepare a 0.25× concentration compared to single-strength noni juice. Green tea extract (60%) was dissolved in ultrapure water, as was grape seed extract, blueberry fruit extract, and curcumin (90%) to the desired concentration. These samples were also combined in equal volumes for tests that involved combinations, resulting in lower concentrations of each ingredient. The samples were mixed with assay buffer (20 mM Tris-HCl, 100 mM NaCl, one mM EDTA, pH 8). Human recombinant DPP4 was then added to this mixture. Afterward, a DPP4 substrate solution comprising 100 μM H-Gly-Pro conjugated aminomethyl coumarin was added. Following incubation at 37° C. for 30 minutes, the fluorescence intensity of each sample reaction was measured with a microplate reader at 360 nm excitation and 460 nm emission. DPP4 enzyme activity was also measured in the same manner by the fluorescence of the initial or uninhibited. The percent difference in fluorescence intensity of the samples versus that of uninhibited DPP4 was used to determine the percent inhibition of the samples.

### Test Results

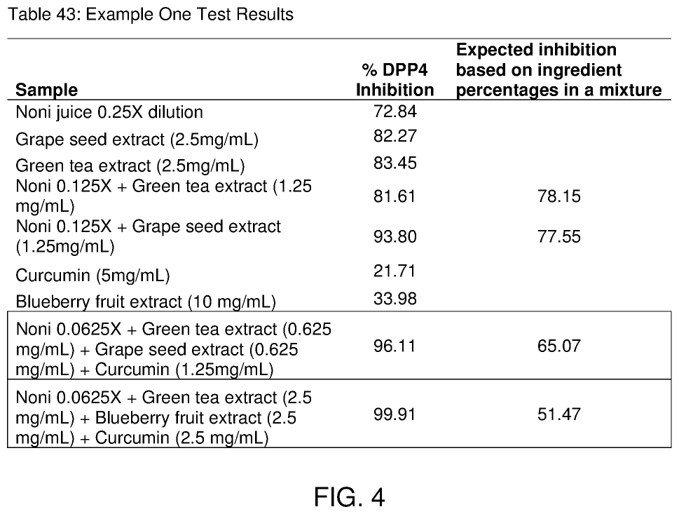

FIG. 4 depicts Table 4, showing that the combination of noni juice and green tea extract provides a slight (3.46%) increase in DPP4 inhibition above expected levels. However, the combination of grape seed extract and noni juice increases DPP4 inhibition by more than 16% above the expected amount. When these ingredients are combined with curcumin, there is a 31.04% increase in enzyme inhibition above expected levels. In particular, the combination of noni juice, green tea extract, curcumin, and blueberry fruit extract almost entirely inhibits DPP4 activity at 99.91%. This reveals particularly strong synergism, as it is 48.44% greater than expected. The collective interaction of all the phytochemicals within each ingredient has a more powerful effect on DPP4 than those from any single ingredient. This is especially noteworthy when considering that the synergy occurs with significantly lower concentrations of each ingredient.

What is claimed is:

1. A formulation for an anti-aging dietary supplement that synergistically inhibits Dipeptidyl Peptidase-4 (“DPP4”) comprising:
Morinda citrifolia fruit;
Camellia sinensis leaf extract;
Curcuma longa root extract containing curcumin; and
Vaccinium corymbosum fruit extract.

2. The formulation of claim 1, further comprising an amino acid, wherein the amino acid comprises L-Arginine, L-Citrulline, beta-alanine, and L-Lysine.

3. The formulation of claim 1, further comprising a plurality of carotenoids.

4. The formulation of claim 1, further comprising melatonin.

5. The formulation of claim 1, further comprising Linum usitatissimum oil.

6. The formulation of claim 1, further comprising Vitis vinifera seed extract.

7. The formulation of claim 1, further comprising:
L-Arginine;
L-Citrulline;
L-Lysine;
beta-alanine;
Vitamin B3 as Niacin;
Vitamin B3 as Niacinamide;
Vitamin B6;
Vitamin B12;
chromium;
magnesium;
selenium;
zinc;
a plurality of carotenoids;
melatonin;
Linum usitatissimum oil; and
Vitis vinifera seed extract.

8. The formulation of claim 1, further comprising, by weight of the formulation:
L-Arginine, L-Citrulline, and L-Lysine in an amount between 120 and 6000 mg;
beta-alanine in an amount between 5 and 250 mg;
Vitamin B3 as Niacin in an amount between 2 and 100 mg;
Vitamin B3 as Niacinamide in an amount between 20 and 1000 mg;
Vitamin B6 in an amount between 0.5 and 25 mg;
Vitamin B12 in an amount between 0.001 and 0.5 mg;
chromium in an amount between 0.005 and 0.25 mg;
magnesium in an amount between 12 and 600 mg;
selenium in an amount between 0.01 and 0.5 mg;
zinc, Vaccinium corymbosum fruit extract, Linum usitatissimum oil, and Camellia sinensis leaf extract in an amount between 1.5 and 75 mg;
a plurality of carotenoids in an amount between 1 and 50 mg;
Curcuma longa root extract comprising curcumin, and Vitis vinifera seed extract, in an amount between 2.5 and 125 mg;
melatonin in an amount between 0.1 and 10 mg; and
Morinda citrifolia fruit in an amount between 10 and 500 mg.

9. The formulation of claim 1, further comprising, by weight of the formulation:
L-Arginine, L-Citrulline, and L-Lysine in an amount between 660 and 3600 mg;
beta-alanine in an amount between 27.5 and 150 m;
Vitamin B3 as Niacin in an amount between 11 and 60 mg;
Vitamin B3 as Niacinamide in an amount between 110 and 600 mg;
Vitamin B6 in an amount between 2.75 and 15 mg;
Vitamin B12 in an amount between 0.0055 and 0.03 mg;
chromium in an amount between 0.0275 and 0.15 mg;
magnesium in an amount between 66 and 360 mg;
selenium in an amount between 0.055 and 0.3 mg;
zinc, Vaccinium corymbosum fruit extract, Linum usitatissimum oil, and Camellia sinensis leaf extract in an amount between 8.25 and 45 mg;
a plurality of carotenoids in an amount between 13 and 37.5 mg;
Curcuma longa root extract comprising curcumin, and Vitis vinifera seed extract, in an amount between 13.75 and 75 mg;
melatonin in an amount between 1.55 and 6.5 mg; and
Morinda citrifolia fruit in an amount between 37.5 and 282.5 mg.

10. The formulation of claim 1, further comprising, by weight of the formulation:
L-Arginine, L-Citrulline, and L-Lysine in an amount equal to 1200 mg;
beta-alanine in an amount equal to 50 mg;
Vitamin B3 as Niacin in an amount equal to 20 mg;
Vitamin B3 as Niacinamide in an amount equal to 200 mg;
Vitamin B6 in an amount equal to 5 mg;
Vitamin B12 in an amount equal to 0.01 mg;
chromium in an amount equal to 0.05 mg;
magnesium in an amount equal to 120 mg;
selenium in an amount equal to 0.1 mg;
zinc, Vaccinium corymbosum fruit extract, Linum usitatissimum oil, and Camellia sinensis leaf extract in an amount equal to 15 mg;
a plurality of carotenoids in an amount equal to 25 mg;
Curcuma longa root extract comprising curcumin, and Vitis vinifera seed extract, in an amount equal to 25 mg;
melatonin in an amount equal to 3 mg; and
Morinda citrifolia fruit in an amount equal to 65 mg.

11. A formulation for an anti-aging dietary supplement that synergistically inhibits DPP4 comprising:
Morinda citrifolia fruit juice;
Camellia sinensis leaf;
Curcuma longa extract; and
Vitis vinifera seed extract.

12. The formulation of claim 11, further comprising an amino acid, wherein the amino acid comprises L-Arginine, L-Citrulline, beta-alanine, and L-Lysine.

13. The formulation of claim 11, further comprising a plurality of carotenoids.

14. The formulation of claim 11, further comprising Gamma-Aminobutyric Acid (GABA).

15. The formulation of claim 11, further comprising Linum usitatissimum oil and Vaccinium corymbosum fruit extract.

16. The formulation of claim 11, further comprising:
L-Arginine;
L-Citrulline;
L-Lysine;
beta-alanine;
Vitamin B3 as Niacin;
Vitamin B3 as Niacinamide;
Vitamin B6;
Vitamin B12;
chromium;
magnesium;
selenium;
zinc;
a plurality of carotenoids;
GABA;
Linum usitatissimum oil; and
Vaccinium corymbosum fruit extract.

17. The formulation of claim 11, further comprising, by weight of the formulation:
L-Arginine, L-Citrulline, and L-Lysine in an amount between 120 and 6000 mg;
beta-alanine in an amount between 5 and 250 mg;
Vitamin B3 as Niacin in an amount between 2 and 100 mg;
Vitamin B3 as Niacinamide in an amount between 20 and 1000 mg;
Vitamin B6 in an amount between 0.5 and 25 mg;
Vitamin B12 in an amount between 0.001 and 0.5 mg;
chromium in an amount between 0.005 and 0.25 mg;
magnesium in an amount between 12 and 600 mg;
selenium in an amount between 0.01 and 0.5 mg;
zinc, Vaccinium corymbosum fruit extract, Linum usitatissimum oil, and Camellia sinensis leaf extract in an amount between 1.5 and 75 mg;
a plurality of carotenoids in an amount between 1 and 50 mg;
Curcuma longa root extract comprising curcumin, and Vitis vinifera seed extract, in an amount between 2.5 and 125 mg;
GABA in an amount between 50 and 2500 mg; and
Morinda citrifolia fruit in an amount between 10 and 500 mg.

18. The formulation of claim 11, further comprising, by weight of the formulation:
L-Arginine, L-Citrulline, and L-Lysine in an amount between 660 and 3600 mg;
beta-alanine in an amount between 27.5 and 150 m;
Vitamin B3 as Niacin in an amount between 11 and 60 mg;
Vitamin B3 as Niacinamide in an amount between 110 and 600 mg;
Vitamin B6 in an amount between 2.75 and 15 mg;
Vitamin B12 in an amount between 0.0055 and 0.03 mg;
chromium in an amount between 0.0275 and 0.15 mg;
magnesium in an amount between 66 and 360 mg;
selenium in an amount between 0.055 and 0.3 mg;
zinc, Vaccinium corymbosum fruit extract, Linum usitatissimum oil, and Camellia sinensis leaf extract in an amount between 8.25 and 45 mg;
a plurality of carotenoids in an amount between 13 and 37.5 mg;
Curcuma longa root extract comprising curcumin, and Vitis vinifera seed extract, in an amount between 13.75 and 75 mg;
GABA in an amount between 275 and 1500 mg; and
Morinda citrifolia fruit in an amount between 37.5 and 282.5 mg.

19. The formulation of claim 11, further comprising, by weight of the formulation:
L-Arginine, L-Citrulline, and L-Lysine in an amount equal to 1200 mg;
beta-alanine in an amount equal to 50 mg;
Vitamin B3 as Niacin in an amount equal to 20 mg;
Vitamin B3 as Niacinamide in an amount equal to 200 mg;
Vitamin B6 in an amount equal to 5 mg;
Vitamin B12 in an amount equal to 0.01 mg;
chromium in an amount equal to 0.05 mg;
magnesium in an amount equal to 120 mg;
selenium in an amount equal to 0.1 mg;
zinc, Vaccinium corymbosum fruit extract, Linum usitatissimum oil, and Camellia sinensis leaf extract in an amount equal to 15 mg;
a plurality of carotenoids in an amount equal to 25 mg;
Curcuma longa root extract comprising curcumin, and Vitis vinifera seed extract, in an amount equal to 25 mg;
GABA in an amount equal to 500 mg; and
Morinda citrifolia fruit in an amount equal to 65 mg.

20. A method for administering an anti-aging dietary supplement that synergistically inhibits DPP4 comprising:
combining Morinda citrifolia fruit, Camellia sinensis leaf extract, Curcuma longa root extract comprising curcumin, and Vaccinium corymbosum fruit extract, to form a dietary supplement; and
administering the dietary supplement daily to inhibit DPP4 activity.
